(19) United States
(12) Patent Application Publication
(10) Pub. No.: US 2024/0027472 A1
(43) Pub. Date: Jan. 25, 2024

(54) METHODS AND DESIGN OF LUNG HEALTH DIAGNOSTIC (LHDx) TECHNOLOGY FOR DIAGNOSIS AND PROGNOSIS-BASED INTERVENTION OF CHRONIC OBSTRUCTIVE PULMONARY DISORDER (COPD), EMPHYSEMA AND AGE-RELATED LUNG DISEASES

(71) Applicant: Neeraj VIJ, Baltimore, MD (US)

(72) Inventor: Neeraj VIJ, Baltimore, MD (US)

(21) Appl. No.: 18/037,047

(22) PCT Filed: Nov. 29, 2021
(86) PCT No.: PCT/US2021/061014

§ 371 Date: May 15, 2023

Related U.S. Application Data
(60) Provisional application No. 63/241,018, filed on Sep. 6, 2021.
(60) Provisional application No. 63/199,007, filed on Dec. 1, 2020.

(51) Int. Cl. G01N 33/68; G01N 33/543

(52) U.S. Cl. CPC G01N 33/6893; G01N 33/54388; G01N 2800/122; G01N 2800/52

(57) ABSTRACT

Methods and design are provided of a lung health diagnostic (LHDx) assay for diagnosis, validation and prognosis-based intervention of chronic obstructive pulmonary disorder (COPD)-emphysema and age-related lung disease in a subject, wherein COPD and age-related lung disease diagnosis comprises generating aggresome positive quantitative data from saliva, respiratory or body fluid sample of the subject by direct quantitative and/or direct analysis including immunoprecipitation and immunoconjugate(s) fluorescence, signal intensity and/or morphological characteristics, (b) obtaining baseline/clinical data or individual risk factors from the said subject and (c) combining the aggresome data with the clinical data or individual risk factors or vice versa to predict, diagnose or validate COPD-emphysema or age-related lung condition in the subject. In addition, a UV LED device is used as a reader for lateral flow assay (LFA) with QD immunoconjugate(s), wherein images of LFA test lines of LFA test strips are captured by an image sensor such as camera or scanner using a smartphone, tablet or point of care (POC) reader device.

### CROSS-REFERENCE TO RELATED APPLICATIONS

This application is based upon and claims the benefit of priority of the prior U.S. Provisional Application Nos. 63/199,007 filed on Dec. 1, 2020 and 63/241,018 filed on Sep. 6, 2021, respectively, the contents of which are incorporated herein by reference.

### BACKGROUND

### 1. Field of Invention

Aspects of the present invention relate to the fields of pulmonary medicine, respiratory health and diagnostics. Specifically, aspects of the present invention relate to methods and design of lung health diagnostic (LHDx) tests, and protype platform technologies for diagnosis and prognosis-based intervention of chronic obstructive pulmonary disorder (COPD), emphysema and age-related lung diseases.

### 2. Description of the Related Art

Chronic obstructive pulmonary disorder (COPD), a leading cause of death worldwide, is triggered by exposure to cigarette smoke (CS) and/or air pollutants and aging. Recent studies have revealed that both CS and aging, two leading causes of COPD-emphysema pathogenesis, can impair “proteostasis” and “autophagy” activities. Thus, proteostasis/autophagy impairment has been verified as a central mechanism for inducing cytosolic/nuclear aggregation of ubiquitinated proteins as “aggresomes-bodies” that triggers chronic oxidative-inflammatory stress, apoptosis, senescence and emphysema progression. In addition, in recent years, electronic-cigarette vaping (eCV) and waterpipe smoking (WPS) have been marketed to smokers as safer alternatives to conventional tobacco cigarette smoking as they use diverse concentrations of nicotine/tobacco combined with a mixture of various flavors, which are either vaporized (e-cigarette) or smoked through a water filter (waterpipe).

However, studies suggest that exposure to vaping and WP tobacco-smoke (or herbal nicotine-free WP-smoke) can also induce significant respiratory toxicity and/or inflammatory-oxidative stress responses, similar to regular CS, suggesting their potential roles in lung cancer and/or COPD-emphysema pathogenesis on chronic exposures. Moreover, environmental exposure to biomass smoke and air pollutants has been identified as a leading cause for COPD in developing countries, along with CS and aging. These issues raise a significant public health concern, as currently there is/are no quantitative bioassay(s) to determine the impact of smoke and air-pollutant exposure, as well as vaping and aging, on lung health to develop guidelines to regulate exposure and effectively treat impacted subjects. It has been validated that in smoke or environmental exposure and/or age-related lung conditions, aggresome is an effective prognostic marker for diagnosis (FIG. 1) and prognosis-based intervention.

### SUMMARY OF THE INVENTION

COPD is the leading cause of death worldwide. COPD has been characterized by impaired breathing or shortness of breath (dyspnea), which is caused by loss of elasticity of pulmonary airway capillaries and air-sacs. Cigarette smoking and vaping, along with exposure to environmental pollutants and aging, are the leading causes of this non-reversible obstructive lung disease, COPD-emphysema. In contrast, asthma is a reversible obstructive respiratory disease, which can be treated using inhaled corticosteroids. Moreover, COPD lacks therapies targeting central disease-causing mechanisms, instead, current therapies are focused on treating symptoms such as using bronchodilators that partially rescue the loss of elasticity of airway capillaries and air-sacs, and are the first line of treatment for COPD together with inhaled antibiotics, mucolytics, etc.

Moreover, COPD lung disease has several associated complications, such as enhanced mucus production and microbial infections that exacerbate the disease state. Thus, having an early diagnosis using a quantitative bioassay, and further treating central disease-causing mechanisms leading to various prognostic symptoms are keys to an ultimate cure. Embodiments of the present invention provide for a prognosis-based intervention strategy that serves the “unmet” clinical need for early diagnosis and treatment, as a companion diagnostic (CDx), using currently available therapeutics or selective novel or emerging interventions to fight COPD and age-related disorders or symptoms. Thus, aspects of the present invention circumvent the use of multiple synchronous treatments that are not only difficult to implement clinically but have poor outcomes, ultimately requiring a lung transplant, due to a late-stage severe COPD-emphysema diagnosis. The possibilities for a more accurate evaluation can provide guidance for earlier and targeted intervention(s).

Briefly, autophagy is a cell's inherent mechanism to engulf and recycle abnormal, malfunctioning and superfluous molecules, such as proteins, lipids or cellular organelle debris. Studies have identified that proteostasis/autophagy impairment is a central mechanism for COPD pathogenesis and progression that is triggered by cigarette or biomass smoke, vaping and aging. It has further been demonstrated that exposure to air pollutants leads to obstructive pulmonary diseases via the same mechanism. Thus, quantitative detection of proteostasis/autophagy-impairment in nasal cells, induced-sputum, saliva or bronchoalveolar lavage fluid (BALF) airway cells or body fluid from subjects at risk (like those exposed to cigarette smoke and air pollution) is an attractive strategy for the early diagnosis of obstructive lungs diseases, particularly in at-risk individuals, such as smokers or inhabitants in high pollutant cities or in elderly subjects. To this end, there has been provided, in accordance with the teachings herein, a novel method and design of diagnostic assay for the detection and quantification of autophagy impairment and/or aggresomes that, as taught here, demonstrates that this assay is able to identify early signs of obstructive lung disease and/or an age-related lung condition.

Aspects of the present invention provide methods for diagnosis of COPD and lung aging related disorders in a subject comprising (a) aggresome-positive data from nasal, BALF or induced-sputum airways cells in a sample or body fluid (BALF supernatant, blood, saliva) obtained from the subject based on direct quantification by multiplex lateral flow assay (LFA) using quantum dot (QD) immunoconjugates, immunofluorescent or chemiluminescent staining, or localization of peri-nuclear aggresomes and/or functional autophagy/proteostasis activity in the sample, wherein aggresomes are identified in the context of peri-nuclear ubiquitinated protein aggregates based on a combination of the immunofluorescence or chemiluminescence staining and/or functional activity assay or markers; (b) obtaining clinical data and/or risk factors for the subject; and (c) combining the aggresome or autophagy/proteostasis activity data with the clinical data and/or risk factors to diagnose risk of COPD and lung aging induced diseases in the subject.

In some embodiments of the present invention, clinical data comprises one or more pieces of spirometry, force oscillation technique (FOT), impulse oscillometry (IOS) or electrical impedance tomography (EIT) based pulmonary function test (PFT) and/or positron emission tomography-computed tomography (PET/CT), X-ray fluoroscopy, CT, or magnetic resonance imaging (MRI) data. The clinical data may be related to one or more individual risk factors such as cigarette smoking or environmental exposure and/or aging. In some instances, the lung disease is COPD-emphysema, where in other instances, the lung disease is an early stage or onset of COPD-emphysema. In other embodiments, the subject is at a high-risk of age-related lung diseases. In these embodiments, the aggresomes data may be generated by fluorescent or chemiluminescent reading of an immunoconjugate probe using an LFA device/reader or scanning via benchtop flow cytometry or microscopy. In further embodiments, immunofluorescent or chemiluminescent staining of peri-nuclear aggresome, p62, CFTR, HDAC6 and/or Ub positive bodies may be performed. In additional embodiments, aggresomes may have distinct immunofluorescence or chemiluminescent staining from surrounding peri-nuclear structures or organelles. In other embodiments, aggresomes comprise distinct morphological characteristics compared to surrounding peri-nuclear bodies. Diagnosis may be expressed as a risk score based on clinical history, smoke exposure (first- and second-hand), genetic predisposition and/or age etc. The risk score, in an embodiment, may be represented as “high”, “low”, etc., or as a numerical number.

Currently, there is no prototype lung health diagnostic in the market that permits the evaluation of the impact of smoking, biomass smoke, air pollutants and vaping or aging on underlying causes of the disease, for early COPD and age-related condition diagnosis and intervention. The current gold standard lung diagnostic modalities include a PFT, lung or chest imaging and biopsy-based diagnostics, which result in significantly late-stage diagnosis of fatal lung conditions. Thus, there are over 16 million cases of undiagnosed COPD in the United States (54%) alone and lot more globally (70%) that are often missed using the current standard of care (SOC) diagnostics, a conventional PFT. Hence, COPD, emphysema and age-related lung disease subjects have late-stage diagnosis, resulting in significant mortality (3 million American deaths/year and 1 death every 2 seconds globally), due to limitations of current interventions in reversing disease from the severe stages.

In addition to early diagnosis, LHDx supports standardization and use of novel, non-invasive, prognosis-based intervention strategy(ies) for COPD-emphysema and age-related lung conditions as a companion diagnostic (CDx). Thus, a foundation has been built for identifying individuals that are highly susceptible to chronic obstructive or age-related lung disease, and resulting failures, for prognosis based early or targeted intervention, to provide a significant societal impact on respiratory health, by improving quality of life (QoL) and Quality of care (QoC), reducing health care costs and mortality.

Focus is on clinical translation of a novel bioassay prototype/platform to specifically quantify pathological aggresome/autophagy-bodies for evaluating the impact of aging and smoke exposure and vaping, etc., on the lungs as well as evaluating the prognosis of COPD-emphysema and age-related disorders. Thus, the LHDx assay prototype/platform supports quantification of (1) aggresome-bodies, (2) autophagy-flux, (3) extracellular ubiquitin or ubiquitinated proteins (EC-Ub) and/or (4) proteostasis activity in the airway cells (induced sputum, nasal swab and/or bronchoalveolar lavage fluid (BALF) or a body fluid (BALF supernatant, blood, saliva etc.)) to quantify initiation and progression of COPD-emphysema and other age-related lung conditions.

Aggresome-bodies, autophagy-flux and proteostasis activity represent potential targets for monitoring disease initiation and progression if they can be quantified non-invasively. Aggresome-bodies have been reported in literature for the past few decades, primarily in neurological pathologies, and their recently described role in lung diseases makes them useful targets for evaluation. Embodiments of the present invention provide techniques which employ an immunomagnetic based antibody platform using VCP/p97-negative selection and capture of p62/Ub-aggresome biomarkers (PTNx) for monitoring disease progression and response to therapeutic or clinical intervention in COPD and lung aging. Embodiments of the present invention disclose clinical utility by standardizing the platform for advanced diagnostic methods. Pre-clinical and clinical datasets have confirmed that aggresome-pathology and autophagy-proteostasis impairment are directly co-related to severity and progression of COPD-emphysema and age-related lung diseases and hence provide an optimal way to quantify therapeutic response for prognosis-based intervention in early stages of the disease. However, quantitative methods for diagnosis have been less promising due to poor detection sensitivity and specificity.

Other less technically sensitive platforms exist that enrich aggresome positive cells by aggresome dye dependent and independent techniques with the ability to detect a ˜5 log fold increase in protein aggregates as aggresomes, which may not necessarily represent pathological peri-nuclear protein-aggregates not able to be cleared by autophagy or proteasomal degradation. To date, aggresome and autophagy/proteostasis assays have not been well studied or developed for risk stratifying COPD, emphysema and lung aging to determine their use as a powerful diagnostic assay.

Other features and advantages of the invention will be apparent from the detailed description.

### BRIEF DESCRIPTION OF THE DRAWINGS

FIG. 1 shows a schematic representation of mechanisms of COPD/respiratory exacerbations and lung disease pathogenesis.

FIG. 2(a) illustrates a vertical view of LHDx flow strips; FIG. 2(b) is a side view of an LHDx flow strip; FIG. 2(c) illustrates an inside view of the components of a lateral flow test strip; FIG. 2(d) illustrates standard vials for solutions such as lysis and elution buffer for sample preparation; FIG. 2(e) illustrates a view of a multiplex lateral flow test cassette in which two test strips are placed; and FIG. 2(f) illustrates a nasal brush for nasal sampling.

FIGS. 3(a) and 3(b) illustrate schematics of sample processing for a multiplex point of care test (xPOCT) and home-based lateral flow assay (LFA) wherein FIG. 3(a) illustrates steps for the xPOCT; and FIG. 3(b) illustrates steps for the home-based LHDx LFA.

FIGS. 4(a)-4(c) illustrate schematic views of an LHDx fluorescence-based multiplex point-of-care test (xPOCT); wherein FIG. 4(a) illustrates a sample loaded onto a sample pad port of two strips embedded in a dual cassette; FIG. 4(b) illustrates once analytes enter a conjugate pad, they are bound by their specific QD-Abs; and FIG. 4(c) illustrate complexes entering a nitrocellulose membrane and binding to their specific Abs located at their respective test lines.

FIGS. 5(a) and 5(b) present a rationale and design of a novel prognosis-based intervention strategy for COPD-emphysema; where FIG. 5(a) shows pathophysiological impact of exposure to tobacco, biomass smoke, aging and/or genetic predisposition; and FIG. 5(b) shows an application of a non-invasive high throughput screening methodology for detecting aggresome-bodies in nasal or airway cells derived from induced-sputum, nasal swab or BALF to quantify COPD in non-smokers or smokers without any clinical signs of the lung disease.

FIGS. 6(a)-6(b) illustrate LEDx fluorescent probe readers, one for home and the other for point of care (POC), wherein FIG. 6(a) shows left, top, perspective side views, of an LEDx^{UVA} device for home-based testing; FIG. 6(b) is a perspective frontside view of an LEDx^{UVR} device for multiplex POC testing (xPOCT); FIG. 6(c) is a rear view of the LEDx^{UVR} device shown in FIG. 6(b), and FIG. 6(d) is a bottom view of the LEDx^{UVR} device shown in FIG. 6(b).

FIG. 7(a) shows a configuration of a system for the LEDx^{UVA} device shown in FIG. 6(a); and FIG. 7(b) shows a configuration for the LEDx^{UVR} device shown in FIGS. 6(b)-6(d).

FIGS. 8(a) and 8(b) show prototype designs of circuit or printed circuit board for the LEDx^{UVA} shown in FIG. 6(a) and the LEDx^{UVR} device shown in FIGS. 6(b)-6(d) for quantum dot excitation and image capture for data analysis.

### DETAILED DESCRIPTION OF THE EMBODIMENTS

Aspects of the present invention are based, in part, on the discovery that adding aggresome and/or autophagy/proteostasis activity data (such as from COPD or respiratory exacerbations as shown in FIG. 1) to existing clinical information and/or subjects risk factors enhances diagnostic accuracy for patients undergoing evaluation for COPD-emphysema, lung aging or predicting initiation of COPD-emphysema and age-related lung disorders. As is described in detail below, the present disclosure demonstrates the integration of personal risk factors, lung function/PFT, imaging and quantification of aggresomes or autophagy/proteostasis impairment as prognostic biomarkers to develop a risk score for predicting pathogenesis and progression of COPD-emphysema and age-related lung disorders.

FIG. 1 illustrates a schematic representation of mechanisms of respiratory exacerbations and lung disease pathogenesis or progression. The inflammatory/pathogenic receptors and cystic fibrosis transmembrane conductance regulator (CFTR) localized in lipid-raft membranes modulate an immune response on viral or bacterial infection of the airway cells. In subjects with a decreased expression of CFTR due to smoke exposure (acquired CFTR dysfunction, COPD), misfolded-CFTR (ΔF508 CFTR, cystic fibrosis, CF), or elderly subjects, an increase in reactive oxygen species (ROS) activity within the cells inhibits the progression of endocytosed viruses and phagocytosed bacteria into phagolysosomes. Furthermore, the ROS resulting from smoke exposure, misfolded/ΔF508 CFTR or age-related changes causes ceramide accumulation within the plasma membrane, and increases TG2 expression, which causes crosslinking of Beclin-1. This Beclin-1 crosslinking results in perinuclear aggresome body formation that further impairs autophagolysosome formation to degrade autophagic cargo and clear infectious pathogens. As a result of this impaired degradation or clearance, the immune response is further impaired, leading to more ROS formation. This ultimately develops into chronic lung disease with recurrent exacerbations and infections. In case of viral infections such as SARS-CoV-2, the virus binds to the ACE2 receptor TMPRSS2 complex, to fuse with the host cell and gain entry for replication. Autophagosome-lysosomal processing is a standard mechanism for clearance of viruses and other pathogen via xenophagy, which when impaired due to smoke or environmental exposure or genetic predisposition and/or aging, results in exacerbation, chronic inflammation, and pathogenesis of severe lung disease.

The detection and quantification of aggresomes and autophagy/proteostasis activity utilize lateral flow strips, loaded on a single or a dual strip cassette. Samples are collected using a nasal brush for nasal sampling, collection vials or tubes with lysis buffer, media, a phosphate buffer saline (PBS)/buffer, etc., for induced-sputum, BALF, nasal/airway cells, saliva and/or other body fluid analyses as described below and/or methods as would be understood by those skilled in the art.

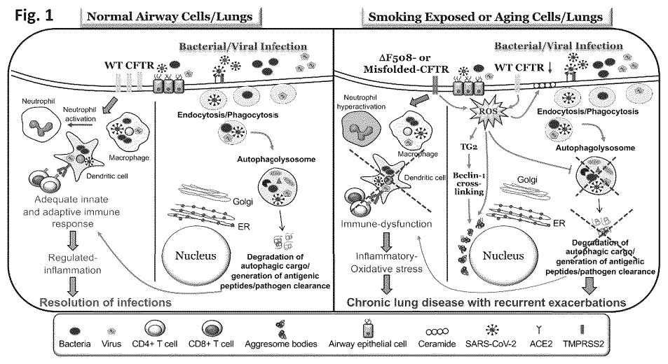

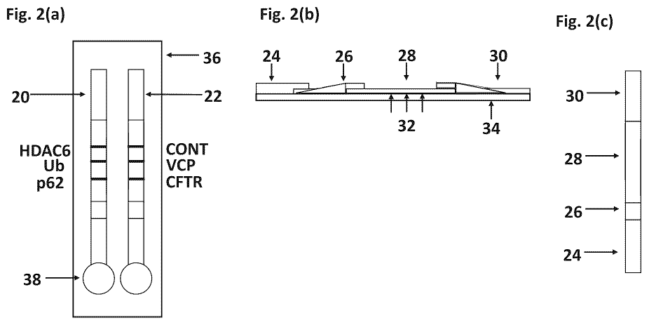

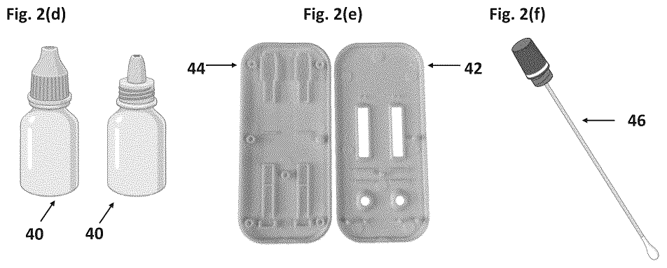

FIGS. 2(a)-2(e) illustrate an LHDx lateral flow assay (LFA) test. FIG. 2(a) illustrates a top view of the LHDx lateral flow test, wherein the test contains two test strips 20, 22. The test strip 20 contains antibodies (Ab) for sequestosome-1 (p62), ubiquitin (Ub), and histone deacetylase 6 (HDAC6), while the test strip 22 contains Abs for cystic fibrosis transmembrane conductance regulator (CFTR), valosin-containing protein (VCP), and a control (IgG) line. As shown in FIGS. 2(b) and 2(c), each test strip 20, 22 contains a sample pad 24, a conjugate pad 26 that holds a quantum dot-Ab (QD-Ab) conjugates, a nitrocellulose membrane 28 with its respective control or test lines 32, and an absorbent pad 30, which are all placed on a plastic backing 34. The test strips 20, 22 are then placed in a housing 36 (FIG. 2(a)) or cassette 44, 42 (FIG. 2(e)) which has a sample port 38 for sample loading.

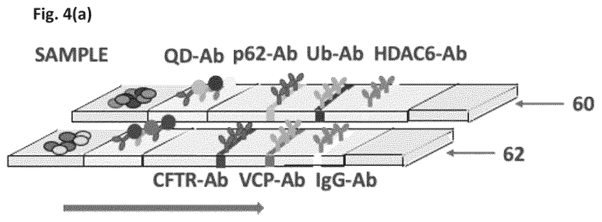

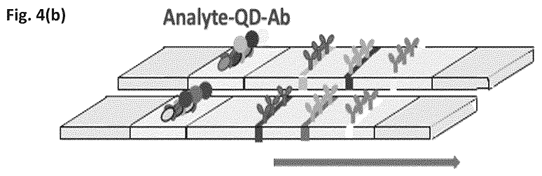

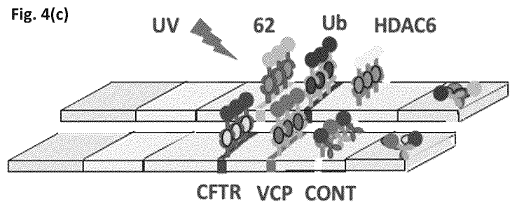

FIG. 2(b) is a side view of the LHDx, and FIG. 2(c) is an inside view of the components of the lateral flow assay (LFA) test. FIG. 2(d) shows standard vials 40 for buffers/solutions such as lysis and elution buffer for sample preparation (to be further explained in FIGS. 4(a)-4(c)). FIG. 2(e) is a base/bottom 44 and top 42 view of the multiplex lateral flow test cassette in which the two test strips 20, 22 are placed inside the cassette base/bottom 44 and cassette top 42. FIG. 2(f) shows a nasal swab 46 for nasal sampling.

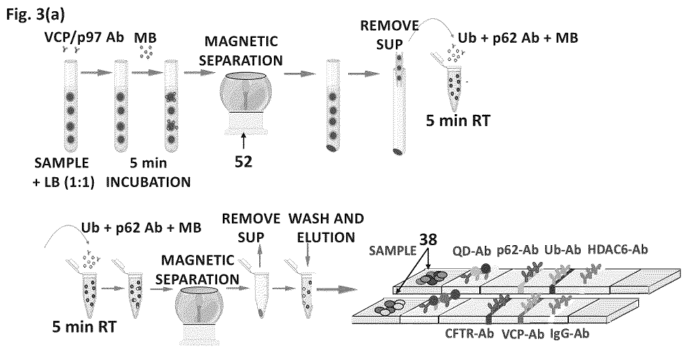

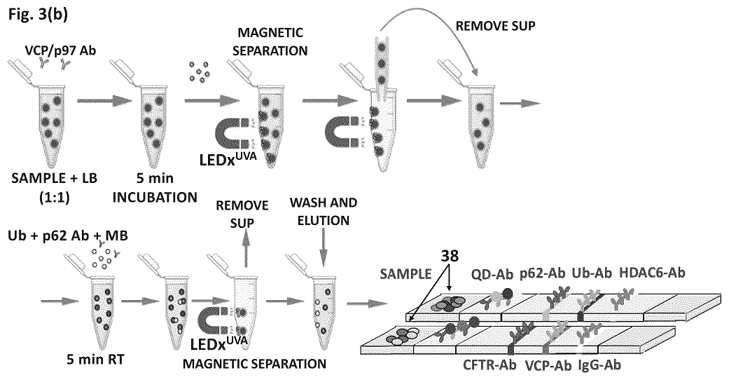

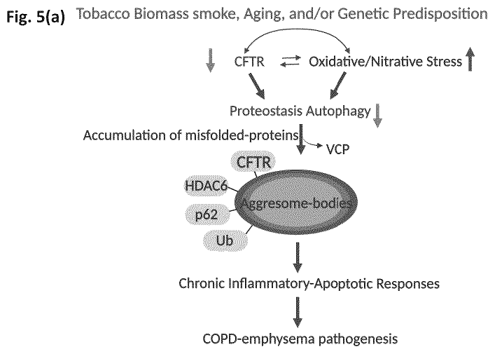

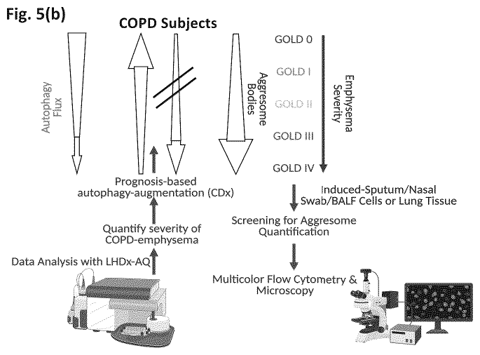

Next, samples are processed for a multiplex point of care test (xPOCT) as shown in FIGS. 4(a)-4(c) by an immunomagnetic lateral flow assay (LFA) as shown in FIGS. 3(a)-3(b) or validated using standard microscopy, flow cytometry or sandwich enzyme linked immunosorbent assay (ELISA) as shown in FIG. 5(b).

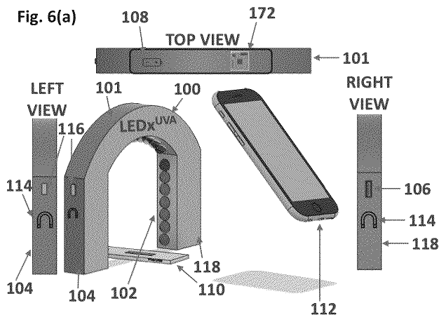

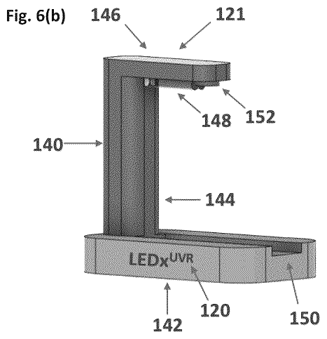

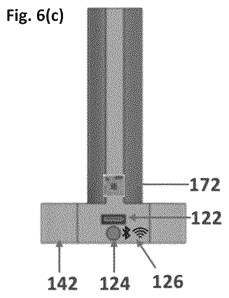

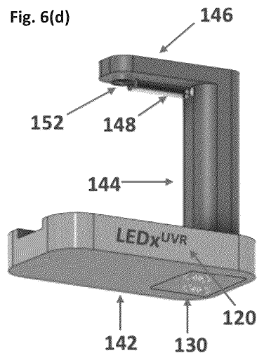

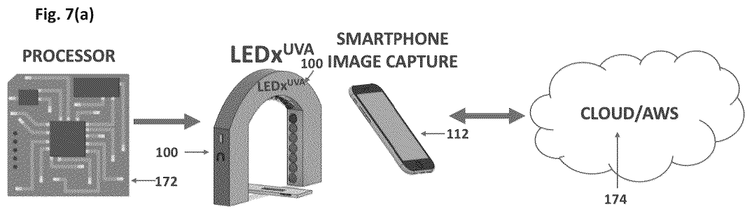

FIGS. 3(a) and 3(b) show steps of sample processing for multiplex point of care test (xPOCT) and home-based lateral flow assay (LFA). Initially, a sample is collected using the nasal brush 46 (FIG. 2(f)). As shown in FIG. 3(a), for the xPOCT, step I involves mixing the collected sample in a lysis buffer (LB) (1:1), incubation with magnetic beads (MB) A/G and valosin-containing protein (VCP)/p97 specific antibodies (Ab), and 5-minute incubation before immunomagnetic depletion of VCP positive cells using a magnetic spinner 52 with magnets, for removal and collection of supernatants for immunomagnetic separation of sequestosome-1-Ubiquitin (p62-Ub) positive aggresome complexes in Step II. FIG. 3(a) also illustrates Step II, where p62-Ub antibodies and MB A/G are incubated for 5-minutes followed by a centrifugal magnetic (using the magnetic spinner 52) concentration of p62/Ub, removal of supernatant (discard or use as a negative control) and 1× washing and elution using washing and elution buffers for immunoprecipitation. The eluted sample are then transferred to the LFA sample port 38 (FIG. 2(a)) to run LFA/diagnostics. FIG. 3(b) shows, for the home-based LHDx LFA, step I involving mixing the collected sample in LB (1:1), incubation with MB A/G and VCP/p97 specific antibodies, and 5-minute incubation before immunomagnetic depletion of VCP positive cells using magnets found inside the LEDx^{UVA} device (shown in FIG. 6a), for removal and collection of supernatants for immunomagnetic separation of sequestosome-1-Ubiquitin (p62-Ub) positive aggresome complexes in Step II. FIG. 3(b) also illustrates Step II, where p62-Ub antibodies and MB A/G are incubated for 5-minutes followed by magnetic separation (using the magnets found inside the LHDx^{UVA} device shown in FIG. 6(a)) concentration of p62/Ub pellet, removal of supernatant (discard or use as a negative control) and 1× washing and elution of pellets using washing and elution buffers. The eluted sample is then transferred to the LFA sample port 38 (FIG. 2(a)) to run the LFA/diagnostics.

With reference to the foregoing, the initial step involves collection of samples in a lysis buffer (1:1) or a media/buffer, etc., (for storage), followed by incubation with magnetic beads A/G and VCP/p97 specific antibodies, for 5 mins to allow immunomagnetic depletion of VCP positive cells using magnetic separation (FIG. 3(a) or 3(b)). The next step involves removal and collection of supernatants for immunomagnetic positive separation of a p62-Ub positive aggresome complex, where p62-Ub antibodies and magnetic beads A/G are incubated for 5 mins followed by a centrifugal magnetic or LEDx^{UVA} based magnetic concentration of p62/Ub+ pellet, removal of supernatant (discard or use as a negative control), followed by 1× washing and elution of pellet using standard washing and elution buffers for immunoprecipitation (FIG. 3(a) or 3(b)).

The eluted sample is loaded on the lateral flow strip (LFS) of a fluorescence or chemiluminescence based multiplex point of care test (xPOCT, FIGS. 4(a)-4(c)).

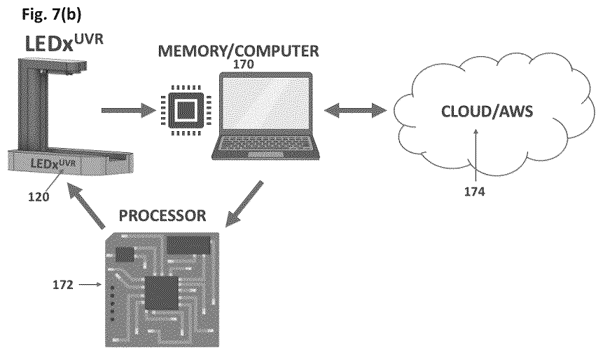

FIGS. 4(a)-4(c) illustrate schematic views of an LHDx fluorescence-based multiplex point-of-care test (xPOCT). As shown in FIG. 4(a), a strip 60 contains antibodies (Ab) for sequestosome-1 (p62), ubiquitin (Ub), and histone deacetylase 6 (HDAC6). A strip 62 contains antibodies for a cystic fibrosis transmembrane conductance regulator (CFTR), a valosin-containing protein (VCP), and a control (IgG) line. After the sample has been prepared as in FIG. 3(a) or 3(b), the sample is loaded onto the sample pad port 38 (as shown in FIG. 2(a)) of the two strips 60, 62 as shown in FIG. 4(a), embedded in the dual cassette 42,44 (as shown in FIG. 2(e)). The conjugate pad 26 (as shown in FIG. 2(b)) is loaded with fluorescently labelled quantum dots (QD) attached to a specific Abs as indicated. The nitrocellulose membrane 28 has another set of Abs for sandwich-based capture of analytes. As shown in FIG. 4(b), once the analytes enter the conjugate pad 26 (as shown in FIG. 2(b)), they are bound by their specific QD-Abs. As shown in FIG. 4(c), these complexes enter the nitrocellulose membrane 28 (as shown in FIG. 2(b)) and bind to their specific Abs located at their respective test lines 32 (as shown in FIG. 2(b)). The different QDs have specific fluorescent bandwidths, leading to QD-fluorescent probe specific signal colors that can be fluoresced by the UV device, LEDx^{UVA} as shown in FIG. 6(a) or LEDx^{UVR} as shown in FIG. 6(b)-(d) and images are captured via LHDx smart-phone app 112 loaded on a smart phone with a camera (shown in FIG. 7(a)) or LEDx^{UVR} with camera (or image sensor) 152 shown in FIG. 7(b) connected to LHDx app or software.

The sample is specifically loaded on the sample pad 24 (as shown in FIG. 2(b)) through the sample port 38 (FIG. 2(a)), of the test strip(s) 20, 22 (as shown in FIG. 2(a), also shown as strips 60, 62 in FIG. 4(a)) embedded in the cassette 42, 44 (as shown in FIG. 2(e)). The conjugate pad 26 (as shown in FIG. 2(b)) of the LFS is loaded with fluorescently labelled quantum dots (QDs) attached to specific antibodies as shown in FIGS. 4(a)-4(c) and the nitrocellulose membrane 28 has another set of antibodies for sandwich-based capture of an aggresome complex attached to Antibody-QD with specific fluorescent bandwidth, leading to QD-fluorescent probe conjugates specific signal colors, that can be read by compatible device or readers as shown in FIGS. 6(a)-6(d). The absorbent pad 30 (as shown in FIG. 2(b)) of the LFS can be used for elution and quantification of unbound QDs, as or if needed.

The laboratory validation tests use 96-well or other plate/slide-based microscopy, flow cytometry or ELISA techniques known to those skilled in art. The combination of aggresome specific immunostaining or capture/immunoprecipitated with p62, Ub, CFTR and/or HDAC6 antibodies followed by morphological analysis of perinuclear (Hoechst/DAPI staining) aggresome bodies and computational quantification of positive fluorescent probe signals using LHDx-A/Q (aggresome quantification) software for lab validation assay(s), as shown in FIGS. 5(a) and 5(b).

FIGS. 5(a) and 5(b) illustrate a rationale and design of an LHDx validation test for a novel prognosis-based intervention of COPD-emphysema where FIG. 5(a) shows exposure to tobacco, biomass smoke, aging and/or genetic predisposition leads to oxidative-nitrative stress that mediates autophagy-impairment initiating aggresome formation, which acts as a central mechanism regulating COPD-emphysema pathogenesis. Thus, aggresome-bodies are implicated in triggering multifarious pathogenic mechanisms such as chronic inflammatory-apoptotic responses that drive the initiation and progression of emphysema in COPD subjects; FIG. 5(b) shows an application of a non-invasive high throughput screening and/or validation methodology for detecting aggresome-bodies in the nasal or airway cells derived from induced-sputum, saliva or BALF to quantify COPD in non-smokers or smokers without any clinical signs of the lung disease. The high throughput flow cytometry and microscopy will assist in rapid screening of multiple samples for the presence and quantification of aggresome-bodies. The data generated from such high throughput assay is analyzed by LHDx aggresome quantification (A/Q) software that assists in determining the severity of aggresome pathology and COPD-emphysema lung disease by quantifying the aggresomes number, morphology, structure, etc. This will allow categorization of subjects into different stages of the disease, based on the levels of aggresomes, which statistically correlates with the lung function decline and COPD-emphysema Global Initiative for Chronic Obstructive Lung Disease (GOLD) stage, I-IV, mild, moderate, severe or very-severe emphysema. Furthermore, the proposed companion diagnostic (CDx), LHDx will be used for prognosis-based personalized intervention utilizing an augmentation or intervention strategy based on the levels of aggresomes that quantify both the extent of proteostasis/autophagy-impairment, and lung function decline. Overall, timely detection and treatment of emphysema or lung function decline by the proposed CDx for prognosis-based intervention strategy will help reduce current mortality rates in this fatal lung condition, as previously discussed.

FIGS. 6(a)-6(d) illustrate PTNx fluorescent lateral flow assay (LFA) probe readers allowing remote, mobile-based or POC readout of fluorescent xPOCT. The prototype LEDx^{UVA} (home-based, 100) and LEDx^{UVR} (POC, 120) devices are used for quantum dot excitation, imaging, and quantification/analysis. FIG. 6(a) illustrates a U-shaped LEDx^{UVA} device 100 for home-based testing to allow reading of the LFA from both sides. The LEDx^{UVA} device 100 contains a U-shaped body 129, with 24 UV LED lights (UV LEDs) 102 having a wavelength of 315-400 nm on the inside of the curvature of the U-shaped body 129 of the LEDx^{UVA} device 100. When the U-shaped body 129 is upside down, a left arm 104 of the U-shaped body 129 contains a power button/switch 116 and a magnet 114. A right arm 118 of the U-shaped body 129 contains a USB power and charging port 106 and another magnet 114. The left arm 104 and the right arm 118 form an internal region of the U-shaped LEDx^{UVA} device 100, wherein the UV LEDs 102 face the internal region as shown. The top 101 of the U-shaped body 129 contains a compartment 108 for a battery to power a printed circuit board (PCB) 172 and the LEDx^{UVA} device 100. The U-shaped body 129 of the LEDx^{UVA} device 100 has arms (left, 104 and right, 118) each of which contain a magnet 114, and the LEDx^{UVA} device 100 is used for sample preparation as shown in FIG. 3(b) for LFA. The magnets 114 are for immunomagnetic separation and the UV LEDs 102 are used for excitation of QD fluorescence. The sample 100 is loaded on the sample port 38 of the LFA as shown in FIGS. 3(a)-3(b). After the sample is run through the LFA, the LEDx^{UVA} device 100 is used as shown in FIG. 6(a) where UV light emitted from the UV LEDs 102 which are mounted on an interior side of the U-shaped body 129 (turned upside down as shown) is utilized to excite the quantum dots on the LFA, resulting in fluorescence emission. The image of the LFA can then be uploaded onto an LEDx app loaded on a mobile device 112 or to any other device with a processor, such as one contained in the LEDx^{UVR} device 120 (shown in FIG. 7(b)). The LEDx app analyzes the image and provides the results of the diagnostics as compared to a baseline for an individual subject to show a positive or negative test result. The LEDx^{UVA} device 100 is used for magnetic separation and excitation of QDs to allow the image capture via the LEDx app and the software.

FIGS. 6(b)-6(d) illustrate an LEDx^{UVR} device 120 for multiplex point of care testing (xPOCT). FIG. 6(b) is a standing upside view of the LEDx^{UVR} device 120 having a body 140 with a base 142, an upright section 144 extending vertically from the base 142, and a top arm 146 extending horizontally from a top end of the upright section 142 and hanging over the base 142. The base 142 has a slot 150 in the top surface. The top arm 146 contains UV LEDs 148 (24 in number in this embodiment), which are located on the underside of the top arm 146 and extending from the upright section 144 to the middle of the top arm 146 having a wavelength of 315-400 nm. The top arm 146, the upright section 144 and the base 142 form an internal region of the LEDx^{UVR} device 120, wherein the UV LEDs 148 face the internal region. FIG. 6(c) is a rear view of the LEDx^{UVR} device 120 shown in FIG. 6(b) and reveals a USB port (USB port and rechargeable battery) 122 and Bluetooth/W-Fi combo PCB 126 for computer connection to a processor 172 (shown in FIGS. 7(a) and 7(b)) and a power button 124 connected to a re-chargeable battery unit 130 in the base 142. FIG. 6(d) illustrates a bottom-up side view of the LEDx^{UVR} device 120. A camera/scanner (or any image sensor) 152 is placed towards the front end of the top arm 146 and the UV LEDs 148 are placed behind the camera/scanner 152, from the rear end to the middle of the top arm (hanging port) 146. UV light emitted from the UV LEDs 148 which are mounted on the underside of the top arm 146 are utilized to excite the quantum dots on the LFA, resulting in fluorescence emission. The camera/scanner 152 takes an image of the LFA cassette.

The LEDx^{UVR} device 120 is used for excitation of QDs and the image capture, where data is transferred via Bluetooth or W-Fi to a tablet, smartphone, or other device for off-site analysis or via a USB cable to a laptop/computer, etc., at a physician's office, clinic or POC.

The LEDx app is stored in a memory which can comprise any combination of cloud (such as AWS, amazon web services), random access memory (RAM), read only memory (ROM), flash memory, cache, static storage such as magnetic or optical disk, or any other types of non-transitory computer-readable media or combinations thereof, which may include a high-speed random access memory (RAM), and may further include a nonvolatile memory such as a magnetic disk storage device, a flash memory device, another volatile solid-state storage device, and the like. The memory may store various operating systems. The memory may be independent and is connected to a processor(s) by using a communications bus; or the memory may be integrated with the processor(s).

The processor(s) 172 may be any type of general or specific purpose processor, including a central processing unit (CPU) or application specific integrated circuit (ASIC), a digital signal processor (DSP), and a field programmable gate array (FPGA). In addition, functional units in the embodiments of the present invention may be integrated into one processing unit 172, or each of the units may exist alone physically, or two or more units are integrated into one unit as shown in FIGS. 7(a) and 7(b).

A person of ordinary skill in the art may understand that all or some of the processes of the methods in the embodiments may be implemented by a computer program, application or software instructing relevant hardware. The program, software and data may be stored in a computer readable storage medium (not shown) or a cloud/AWS 174. When the program is executed, the procedures of the methods in the embodiments are performed. The foregoing storage medium includes any medium that can store program code, such as a random-access memory (RAM), a read-only memory (ROM), a non-volatile RAM (NVRAM), a programmable ROM (PROM), an erasable PROM (EPROM), an electrically erasable PROM (EEPROM), a flash memory, an optical memory, and a register.

FIG. 7(a) shows a configuration of a system for an LEDx^{UVA} device 100, and FIG. 7(b) shows a configuration for an LEDx^{UVR} device 120. As shown in FIGS. 6(a) and 7(a), for the LEDx^{UVR} device 100, after pressing the power button/switch 116, the processor 172 turns on the 24 UV LEDs 102, exciting the quantum dots (QD) on the lateral flow assay (LFA). A user's smartphone 112, tablet or similar device utilizes an LEDx app to capture an image of the LFA via a camera and upload it to the HIPPA complaint cloud/Amazon Web Service (AWS) 174 for storage and quantification using LEDx software. The results are sent back to the smartphone 112 or tablet app and displayed to the user. As shown in FIGS. 6(b)-(d) and 7(b), for the LEDx^{UVR} device 120, it can be connected to the laptop, tablet, or computer 170 via a USB/USB-C cable or Bluetooth/Wi-Fi, which signals the processor 172 through LEDx software to first turn on the UV LEDs 148 to excite QDs on the LFA strip(s), that is inserted in the base on LFA cassette slot of the LEDx^{UVR}. Next LEDx software can be used to activate image capture of the LFA using the LEDx^{UVR} CCD camera/scanner 152. The image is then sent back to an app or software on the tablet/computer 170, which uploads it to the HIPAA compliant cloud/Amazon Web Service (AWS) 174 for storage and quantification using LEDx software. The results are sent back to the app or software on the tablet/computer 170, which can store the image/results on a local drive as well.

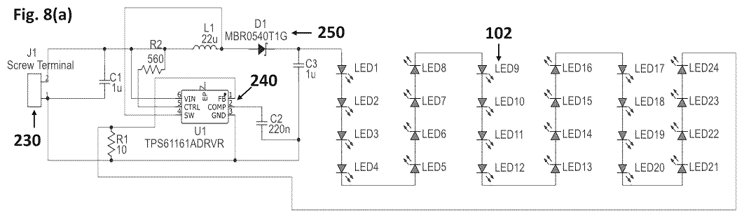

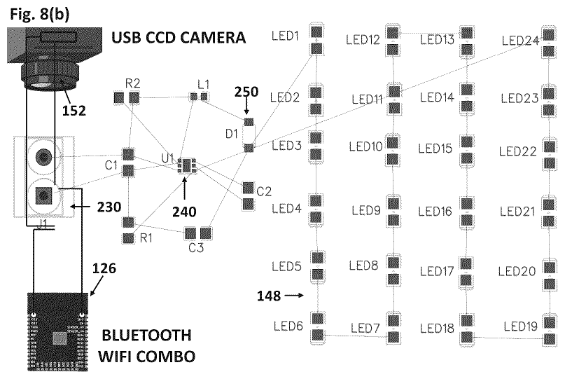

FIGS. 8(a) and 8(b) show designs of the prototype LEDx^{UVA} and LEDx^{UVR} devices 100, and 120 circuits for a printed circuit board (PCB), respectively, for quantum dot excitation. As shown in FIG. 8(a), a circuit powers the 24 UV LEDs 102 (LED1-LED24) for LEDx^{UVA}. The circuit or PCB design includes a screw terminal (J1) 230, a TPS61161A LED lighting driver (U1) 240, Schottky Power Rectifier (D1) 250, 21 μF capacitors (C1, C3), a 220 nF capacitor (C2), a 10 Ohm resistor (R1), a 560 Ohm resistor (R2), a 220 pH inductor (LI), and the 24 UV LEDs 102 (LED1-24). A DC voltage is provided through the screw terminal J1. The LED lighting driver U1 is a boost converter that drives the UV LEDs 102 in series and allows for the UV LEDs 102 to continuously glow with maximum brightness.

FIG. 8(b) shows an LED activation circuit for a printed circuit board (PCB) design for LEDx^{UVR}, where the circuit powers the 24 UV LED lights 148 (LED1-LED24) for LEDx^{UVR}. The design includes a screw terminal (J1) 230, a TPS61161A LED lighting driver (U1) 240, Schottky Power Rectifier (D1) 250, 21 μF capacitors (C1, C3), a 220 nF capacitor (C2), a 10 Ohm resistor (R1), a 560 Ohm resistor (R2), a 220 pH inductor (LI), and the 24 UV LEDs 148 (LED1-LED24). A DC voltage is provided through the screw terminal J1. The LED lighting driver U1 is a boost converter that drives the UV LEDs 148 in series and allows for the UV LEDs 148 to continuously glow with maximum brightness. Text next to each component corresponds to components in the circuit sketch and other components that include the USB CCD camera/scanner 152 and Bluetooth/Wi-Fi Combo PCB 126 that are all power sourced through the USB/USB-C port 122 (shown in FIG. 6(a)) and rechargeable battery 130 (shown in FIG. 6(d)).

Aspects of the present invention provide a method for diagnosing COPD-emphysema in a subject comprising (a) generating aggresome positive (using methods and test components described in FIGS. 2(a)-4(c) and/or 5(b)) and/or (b) autophagy/proteostasis activity quantitative data from a sputum, nasal brushing/swab, BALF airway cell or body fluid (BALF supernatant, blood, saliva) sample obtained from the subject, based on a direct analysis using immunofluorescent or chemiluminescent staining, testing functional activity and/or morphological characteristics of perinuclear aggresome-bodies in positive cells of the sample (FIGS. 5(a)-5(b) validation test or FIGS. 6(a)-6(b), home-based [LEDx^{UVA}] or POC [LEDx^{UVR}]), wherein aggresomes are identified in the context of peri-nuclear bodies based on a combination of the immunofluorescent or chemiluminescent staining, functional activity and/or morphological characteristics, (c) obtaining clinical data and/or individual risk factors for the subject, and (d) combining the aggresome and/or autophagy/proteostasis activity data with the clinical data or individual risk factors to diagnose COPD-emphysema and/or an age-related condition in the subject.

Aspects of the present invention provide a method for diagnosing “early-stage” COPD-emphysema in a subject comprising (a) generating aggresome positive (using methods and test components described in FIGS. 2(a)-4(c) and/or 5(b)) and/or (b) autophagy/proteostasis activity quantitative data from a sputum, nasal brushing/swab, BALF, blood, saliva or airway cell sample obtained from the subject, based on a direct analysis using immunofluorescent or chemiluminescent staining, functional activity and/or morphological characteristics of perinuclear aggresome-bodies in positive cells of the sample (FIGS. 5(a)-5(b), validation test or FIGS. 6(a)-6(b), home-based [LEDx^{UVA}] or POC [LEDx^{UVR}]), wherein aggresomes are identified in the context of peri-nuclear bodies based on a combination of the immunofluorescent or chemiluminescent staining, functional activity and/or morphological characteristics, (c) obtaining clinical data and/or individual risk factors for the subject, and (d) combining the aggresome and/or autophagy/proteostasis activity data with the clinical data or individual risk factors to diagnose “early-stage” COPD-emphysema in the subject.

Aspects of the present invention provide a method for diagnosing initiation or progression of age-related lung disease or disorder in a subject comprising (a) generating aggresome positive (using methods and test components described in in FIGS. 2(a)-4(c) and/or 5(b)) and/or (b) autophagy/proteostasis activity quantitative data from a sputum, nasal brushing/swab, BALF, blood, saliva or airway cell sample obtained from the subject, based on a direct analysis using immunofluorescent or chemiluminescent staining, functional activity and/or morphological characteristics of perinuclear aggresome-bodies in positive cells of the sample (FIGS. 5(a)-5(b), validation test or FIGS. 6(a)-6(d), home-based [LEDx^{UVA}] or POC [LEDx^{UVR}]), wherein aggresomes are identified in the context of peri-nuclear bodies based on a combination of the immunofluorescent or chemiluminescent staining, functional activity and/or morphological characteristics, (c) obtaining clinical data or individual risk factors for the subject, and (d) combining the aggresome and/or autophagy/proteostasis activity data with the clinical data or individual risk factors to diagnose initiation or progression of age-related lung disease or disorder in the subject.

Aspects of the present invention provide a method for diagnosing initiation or progression of COPD in a subject comprising (a) generating aggresome positive (using methods and test components described in FIGS. 2(a)-4(c) and/or 5(b)) and/or (b) autophagy/proteostasis activity positive quantitative data from a sputum, nasal brushing/swab, BALF, blood, saliva or airway cell sample obtained from the subject, based on a direct analysis using immunofluorescent or chemiluminescent staining, functional activity and/or morphological characteristics of perinuclear aggresome-bodies in positive cells of the sample (FIGS. 5(a)-5(b), validation test or FIGS. 6(a)-6(d), home-based [LEDx^{UVA}] or POC [LEDx^{UVR}]), wherein aggresomes are identified in the context of peri-nuclear bodies based on a combination of the immunofluorescent or chemiluminescent staining, functional activity and/or morphological characteristics, (c) obtaining clinical data and individual risk factors for the subject, and (d) combining the aggresome data with the clinical data or individual risk factors to diagnose initiation or progression of COPD-emphysema in the subject.

It is noted that, as used in this specification and the appended claims, the singular forms “a”, “an” and “the” also include plural references unless the content clearly dictates otherwise and are used interchangeably with “at least one” and “one or more”. Thus, for example, reference to “a biomarker” can include an aggresome-formation, ubiquitinated-protein and p62, CFTR and/or HDAC6 accumulation or a mixture of two or more such prognostic or predictive biomarkers, and the like.

“A plurality of” refers to two or more than two of something. The terms “and/or” and “at least one of . . . or . . . ” describe an association relationship between associated objects and indicates that any of three relationships may exist. For example, only A exists, both A and B exist, and only B exists.

The term “about,” particularly in reference to a given quantity, is meant to encompass deviations of plus or minus five percent.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but can include other elements not expressly listed or inherent to such process, method, product-by-process, or composition of matter.

The term “subject,” as used herein includes humans as well as other mammals. It is noted that, as used herein, the terms “organism,” “individual,” “subject,” or “patient” are used as synonyms and interchangeably.

As used herein, the terms “ubiquitinated protein aggregates”, “aggresomes” and/or “autophagy/proteostasis activity” are meant to encompass any cell that is present in a biological sample that is related to COPD-emphysema and/or age-related lung disorder.

In its broadest sense, a biological sample can be any sample that contains aggresomes and/or autophagy/proteostasis activity. A sample can comprise a bodily fluid such as blood, saliva, sputum, nasal or BALF; the soluble fraction of a cell preparation, or an aliquot of media in which cells are grown; an organelle, or membrane isolated or extracted from a cell; genomic DNA, RNA, or cDNA in solution or bound to a substrate; a cell; a tissue; a tissue print; a fingerprint; cells; skin, and the like. A biological sample obtained from a subject can be any sample that contains cells or their components of body fluids and encompasses any material in which aggresomes and/or autophagy/proteostasis activity can be detected. A sample can be, for example, BALF, whole blood, plasma, sputum, nasal, saliva or other bodily fluid or tissue that contains cells or their components.

The biological sample may be nasal or airway cells, induced-sputum, saliva, nasal brushing/swab, BALF or blood. As described herein, a sample is more particularly a cell fraction, and still more particularly a cell fraction containing aggresome-bodies and/or autophagy/proteostasis activity. As will be appreciated by those skilled in the art, a sample can include any fraction or component of an airway, without limitation, epithelial, endothelial, T-cells, monocytes, neutrophiles, erythrocytes, platelets, and macrovesicles such as exosomes and exosome-like vesicles. In the context of this disclosure, airway cells include in a BALF, nasal, airway, saliva, or sputum sample that encompass any cells and are not limited to components of nasal, airway, sputum or BALF cells. As such, nasal, airway, saliva, sputum or BALF cells include, for example, epithelial, inflammatory, endothelial, and other circulating cells. A sample can also be body fluid like BALF-supernatant or blood or its components.

The samples of this disclosure can each contain a plurality of cell populations and cell subpopulations or body fluid (BALF supernatant, blood, etc.) that are distinguishable by methods well known in the art (e.g., FACS, ELISA, immunohistochemistry, microscopy etc.). For example, a nasal brushing/swab, induced sputum and BALF airway cell, saliva, or other body fluid (BALF supernatant, blood) sample can contain populations of inflammatory and epithelial/endothelial cells or RBC/erythrocyte, etc. By way of example, the samples of this disclosure are non-enriched samples, i.e., they are not enriched for any specific population or subpopulation of nucleated cells or free extracellular proteins. For example, non-enriched nasal, airway, sputum, or BALF cell or body fluid (BALF supernatant, blood, saliva) samples as collected are not enriched for aggresome positive cells and/or autophagy/proteostasis activity, epithelial, endothelial, B-cells, T-cells, NK-cells, monocytes, or the like.

In some embodiments the sample is a nasal brushing/swab, airway, sputum or BALF cells or body fluid (BALF supernatant, blood, saliva etc.) sample obtained from a healthy subject or a subject deemed to be at high risk of lung diseases based on art known clinically established criteria including, for example, smoking history and age. According to some embodiments, a nasal or airway cell containing a sample or body fluid (BALF supernatant, blood) is from a subject who has been diagnosed with lung disease or symptoms based on lung imaging, biopsy, and/or surgery or clinical grounds. In some embodiments, the nasal, airway, sputum or BALF cell sample or body fluid (BALF supernatant, blood, saliva, etc.) may be obtained from a subject showing a clinical manifestation of lung disease well known in the art or who presents with any of the known risk factors for COPD-emphysema and age-related lung disease. The term “high risk” as used herein in the context of a subject's predisposition for COPD-emphysema means current or recent smokers aged 40 or older with a pack-year history of 20 pack-years or more. Thus, as is understood by those skilled in the art, pack-year is a measure of how much an individual has smoked. For example, one pack-year of smoking corresponds to smoking one package of cigarettes (20 cigarettes) daily for one year. High risk also can refer to an individual exposed to biomass smoke, first- or second-hand cigarette smoke, e-cigarette vapor (eCV), wildfires, air/environmental or industrial pollution, etc., or age-related changes or other genetic predispositions such as gene mutations, single nucleotide polymorphism (SNP), etc.

As used herein in the context of generating aggresome positive and/or autophagy/proteostasis activity data, the term “direct analysis” refers to the aggresomes and/or autophagy/proteostasis activity being quantified in the context of all aggregated proteins or peri-nuclear aggregate bodies present in the sample as opposed to enrichment of the sample for aggresomes prior to magnetic immunoprecipitation or isolation for detection and quantification.

An aspect of the present disclosure is the robustness of the disclosed methods with regard to the detection and quantification of aggresomes and/or autophagy/proteostasis activity. The rapid and early detection and quantification disclosed herein with regard to aggresomes and/or autophagy/proteostasis activity are based on a direct analysis of a cell population that encompasses the identification of rare events in the context of the surrounding non-rare events. Identification of the early events according to the disclosed methods inherently identifies the surrounding events as acute events. Taking into account the surrounding events and determining the averages for such events, for example, average aggresome size and/or or punta-bodies, allows for calibration of the detection method by removing noise. This results in robustness of the disclosed methods that cannot be achieved with methods that are not based on direct analysis and specific selection of aggresomes.

Aspects of the present invention provide methods for detecting aggresomes in nasal, airway, sputum or BALF cell or body fluid (BALF supernatant, blood, saliva, etc.) samples and integrating aggresome and/or autophagy/proteostasis activity data with individual patient risk factors, lung function/PFT and/or imaging data to develop a risk score for predicting lung disease in patients with COPD or GOLD stage I-IV emphysema and/or age-related lung disease. The integration of aggresome and/or autophagy/proteostasis activity data with individual patient risk factors and imaging data significantly augments the use of individual patient risk factors, lung function and imaging data alone for risk stratifying patients undergoing an evaluation for lung disease, and thus provides a transformative non-invasive biomarker technology for diagnosing early-stage COPD-emphysema and age-related lung diseases. In some embodiments, the COPD is GOLD stage-I emphysema. In other embodiments COPD is advance stage (GOLD II-IV), evaluating disease progression or severity.

As used herein, the term “clinical data” encompasses lung function and imaging data and individual risk factors.

The term “lung function data” or “PFT data” or “functional data”, as used herein, refers to any data generated via clinical spirometry/PFT, force oscillation technique (FOT), impulse oscillometry (IOS) or electrical impedance tomography (EIT) based lung function analysis or functional lung imaging of a subject's lung and integrated with other data to diagnose lung function decline or the disease, for example, COPD-emphysema, in a subject, according to the methods used by those skilled in the art.

The term “imaging data” or “lung imaging data” as used herein, refers to any data generated via clinical imaging of a subject's lung and integrated with other data to diagnose lung disease, for example, COPD-emphysema, in a subject according to the methods used by those skilled in the art. As such, the term includes data generated by any form of imaging modality known and used in the art, for example and without limitation, by chest X-ray or X-ray fluoroscopy and lung computed tomography (CT), lung ultrasound, positron emission tomography (PET), electrical impedance tomography and magnetic resonance imaging (MRI). It is understood that one skilled in the art can select lung imaging data based on a variety of art known criteria. As described herein, the methods of aspects of the invention can encompass one or more pieces of imaging data.

Lung imaging data can be generated through the use of any imaging modality known and used by those skilled in the art. Commonly used imaging modalities include chest radiograph, X ray fluoroscopy, computed tomography (CT), scanning and/or magnetic resonance imaging (MRI), positron emission tomography (PET) scanning, etc. In some cases, the lung imaging data is generated using a positron emission tomography-computed tomography (PET/CT) scan. In some embodiments, the PET/CT is a 2-[18]-F-fluoro-2-deoxy-D-glucose (FDG) PET/CT (FDG PET/CT). While exemplified herein with an in-vivo glycolytic marker FDG, or a Quantum Dot (QD)-aggresome prognostic biomarker(s), or any other marker for imaging can be selected by the skilled person in the art to practice the aspects of the present invention methods for imaging and/or lung function analysis.

As described herein, the clinical data generated and utilized in the embodiments of methods of the present invention can encompass one or more pieces of individual risk factors. As used herein, the term “individual risk factor” or “individual risk biomarker” refers to any measurable characteristic in a subject of the change and/or the detection of which can be correlated with COPD-emphysema and integrated with other data to diagnose lung disease, for example, early-stage emphysema in the subject according to the methods known to those skilled in the art. In the methods disclosed herein, one or more individual risk factors can be selected from the group consisting of age, gender, ethnicity, lung disease history, genetic predisposition, lung function decline and/or smoking status. It is understood that one skilled in the art can select additional individual risk factors based on a variety of art known criteria. As described herein, aspects of the methods of the present invention can encompass one or more individual risk factors.

In the methods disclosed herein, aggresome and/or autophagy/proteostasis activity data and clinical data comprise measurable features. Measurable features useful for practicing the methods disclosed herein include any predictive or prognostic biomarker that can be correlated, individually or combined with other measurable features, with early-stage COPD-emphysema in a subject. Such biomarkers can include imaging data, individual risk factors, lung function and aggresome positive and/or autophagy/proteostasis activity data. The aggresome and/or autophagy/proteostasis activity data can include morphological features, functional data and/or immunofluorescent or chemiluminescence features. As will be understood by those skilled in the art, biomarkers can include a biological molecule, or a fragment of a biological molecule, the change and/or the detection of which can be correlated, individually or combined with other measurable features, with early-stage COPD-emphysema in a subject. Biomarkers also can include, but are not limited to, biological molecules comprising nucleotides, nucleic acids, nucleosides, amino acids, sugars, fatty acids, steroids, metabolites, peptides, polypeptides, proteins, carbohydrates, lipids, hormones, antibodies, regions of interest that serve as surrogates for biological macromolecules and combinations thereof (e.g., glycoproteins, ribonucleoproteins, lipoproteins) as well as portions or fragments of a biological molecule.

Aggresomes, which can be present in a single cell or in clusters of cells, are often epithelial cells shed from airway or inflammatory cells and are present in very low concentrations in the nasal, airway, sputum or BALF cell samples of the subject. Accordingly, detection of aggresomes in a nasal, airway, sputum or BALF cell sample can be referred to as rare event detection, where aggresome, autophagy/proteostasis or immunoproteasome activity can be present in respiratory sample, BALF, blood or other body fluids.

The samples of this disclosure may be obtained by any method, including, e.g., by brushing the solid tissue, biopsy or fluid biopsy. A nasal, airway, sputum or BALF cell, or body fluid (BALF supernatant, blood, saliva etc.) sample may be extracted from any source known to include airway or inflammatory cells or components thereof, such as membranes, organelles, and the like. The airway cell-containing, or body fluid samples may be processed using well known and routine clinical methods (e.g., procedures for drawing and processing cells or blood). In some instances, a nasal, airway, sputum or BALF cell or body fluid (BALF supernatant, blood, saliva etc.) sample is drawn into collection tubes, which may contain media, PBS, a protein lysis buffer, ethylenediaminetetraacetic acid (EDTA), blood collection tubes or Cell-Free DNA. In other embodiments, a nasal, airway, sputum or BALF cell or body fluid (BALF supernatant, blood, saliva etc.) sample is drawn into nasal or bronchial brushing or CellSave® tubes. A nasal, airway, sputum or BALF cell or body fluid (BALF supernatant, blood) sample may further be stored for up to 3 hours, 6 hours, 12 hours, 24 hours, 36 hours, 48 hours, 60 hours or 72 hours before further processing.

In some embodiments, the methods of this disclosure comprise an initial step of lysing cells in the nasal, airway, sputum or BALF cell or supernatant sample or processing of blood or saliva samples using standard lab protocols. The cells may be lysed, e.g., by adding a protein lysis buffer to the nasal, sputum or BALF sample or blood or saliva collection using standard lab protocols. In some embodiments, a nasal, airway, sputum or BALF cell or body fluid (BALF supernatant, blood, saliva, etc.) sample is subjected to centrifugation, quick spin, or magnetic separation following cell lysis and centrifuged, immunocaptured aggresome positive cells or pellet/beads are resuspended, e.g., in an elution buffer or PBS solution (FIGS. 3(a)-3(b)).

In some embodiments, nucleated cells from a sample, such as a nasal, airway, sputum or BALF cell sample or body fluid (BALF supernatant, blood, saliva, etc.), are deposited as a monolayer on a planar support, as known to those skilled in art. The planar support may be of any material, e.g., any fluorescently clear material, any material conducive to cell attachment, any material conducive to the easy removal of cell debris, or any material having a thickness of <100 μm. In some embodiments, the material may be a film, a glass slide or microfluidic platform. In some embodiments, the method uses an initial step of depositing nucleated cells from the sample as a monolayer on a glass slide or microfluidic platform. The glass slide or microfluidic platform can be coated to allow maximal retention of live cells. In some embodiments, about 0.1 million, 0.5 million, 1 million, 1.5 million, 2 million, 2.5 million, 3 million, 3.5 million, 4 million, 4.5 million, or 5 million nucleated cells are deposited onto the glass slide. In some embodiments, the methods of this disclosure involve depositing about 0.01 million cells onto a glass slide or microfluidic platform. In some embodiments, the methods of this disclosure comprise depositing between about 0.001 million and about 0.003 million cells onto the glass slide or microfluidic platform. In some embodiments, the glass slide or microfluidic platform and immobilized cellular samples may be available for further processing or experimentation after the methods of this disclosure have been completed.

In some embodiments, the methods of this disclosure may include an initial step of identifying nucleated cells in the nasal, airway, sputum or BALF cell sample or body fluid (BALF supernatant, blood, saliva, etc.). In some embodiments, the peri-nucleated bodies in cells are identified with a fluorescent or chemiluminescent stain. In some embodiments, the fluorescent or chemiluminescent stain comprises a nucleic acid specific stain. In some embodiments, the fluorescent stain is a Hoechst dye or diamidino-2-phenylindole (DAPI) and an aggresome dye such as nile red, spyro orange, other available specific dyes, or BODIPY probes, nano or quantum dot probes. In some embodiments, immunofluorescent staining of nucleated cells comprises aggresome, p62, Ub, CFTR, HDAC6 and/or nuclei (Hoechst/DAPI). In some embodiments further described herein, aggresomes based on its morphological characteristics and peri-nuclear location.

Aggresomes comprise distinct immunofluorescent or chemiluminescent staining of peri-nuclear bodies in the cells. In some embodiments, the distinct immunofluorescent or chemiluminescent staining of aggresomes comprises Hoechst/DAPI (+) surrounding, p62 (+), Ub (+), HDAC6 (+), and/or CFTR (+) punta-bodies that are VCP/p97 (−). The identification of aggresomes further involves comparing the intensity of aggresome fluorescent staining in peri-nuclear space. In some embodiments, the aggresome data may be generated by fluorescent or chemiluminescent scanning microscopy, flow cytometry (FIG. 5(b), validation test) or visual/analytical LFA readout (FIG. 6(a), home-based [LEDx^{UVA}] or FIGS. 6(b)-(d), POC [LEDx^{UVR}]) to detect immunofluorescent staining of peri-nuclear punta-bodies in cells obtained from a nasal, airway, sputum or BALF cell sample.

Aggresomes, which can be present in cells, as single or in clusters of aggresomes, are often seen in epithelial cells shed from the airway or in the inflammatory cells, and they are found in very low concentrations in the nasal, airway, sputum or BALF cell samples of patients. As used herein, the term “cluster” refers to aggresomes or punta-bodies of perinuclear ubiquitinated or aggregated proteins and lipids, while extracellular ubiquitin or immunoproteasome activity is present in body fluid, such as BALF, saliva or blood's non-cellular fractions.

In some embodiments, all peri-nucleated bodies in cells are retained and/or chemiluminescent or immunofluorescent stained with polyclonal or monoclonal antibodies targeting p62 (+), Ub (+), HDAC6 (+), CFTR (+) and VCP/p97 (−), and a nuclear stain, Hoescht/DAPI. The peri-nuclear aggresome positive cells can be imaged in multiple fluorescent channels to produce high quality and high-resolution digital images that retain fine cytologic details of nuclear contour and cytoplasmic distribution (FIGS. 4(a)-5(b)]).

In some embodiments, the aggresome data includes high definition aggresome (HD-aggresome) detection. HD-aggresomes are HDAC6 positive, VCP negative, contain an intact punta-bodies surrounding Hoechst/DAPI positive nucleus with or without identifiable apoptotic changes or a disrupted appearance, and are morphologically distinct from surrounding organelles. Hoescht/DAPI (+) nucleus, and surrounding p62, Ub, HDAC6 and CFTR positive (+, aggresomes) that are VCP/p97 negative (−), where signal intensities can be categorized as measurable features during HD-aggresome detection as previously described, where FIG. 1 describes the mechanism for respiratory exacerbations, and disease pathogenesis and FIGS. 5(a)-5(b) show levels of aggresomes are directly correlated to severity and/or presence of COPD-emphysema. The direct analysis employed by the methods disclosed herein results in high sensitivity and high specificity for validation assay (FIGS. 4(a)-5(b)), while adding high definition cytomorphology to enable detailed morphologic characterization of an aggresome-positive cell population known to be heterogeneous.

While aggresomes can be identified as peri-nuclear bodies comprising p62/Ub/HDAC6/CFTR (+) and VCP (−) aggresomes surrounding a Hoescht/DAPI (+) nucleus, the methods of the present invention can be practiced with any other predictive or prognostic biomarkers that one of skill in the art selects for generating aggresome data and/or identifying aggresomes and aggresome+clusters (FIG. 1). One skilled in the art would understand how to select a morphological feature, biological molecule, or a fragment of a biological molecule, the change and/or the detection of which can be correlated with an aggresome as described above.

A person skilled in the art will appreciate that a number of methods can be used to generate aggresome and/or autophagy/proteostasis activity data, including microscopy based approaches (FIG. 5(b)), including fluorescence scanning microscopy, LFA (lateral flow assay), flow cytometry, chip-based assay, mass spectrometry approaches, such as MS/MS, LC-MS/MS, multiple reaction monitoring (MRM) or selected reaction monitoring (SRM) and product-ion monitoring (PIM) and also including antibody based methods such as immunofluorescence, chemiluminescence, immunohistochemistry, immunoassays, Western blots, enzyme-linked immunosorbent assay (ELISA), immunoprecipitation, radioimmunoassay, dot blotting, and FACS (fluorescence activated cell sorting). Immunoassay techniques and protocols are generally known to those skilled in the art. A variety of immunoassay techniques, including competitive and non-competitive immunoassays, can be used.

A person of skill in the art will further appreciate that the presence or absence of predictive or prognostic biomarkers may be detected using any class of marker-specific binding reagents known in the art, including, e.g., antibodies, aptamers, fusion proteins, such as fusion proteins including protein receptor or protein ligand components, or biomarker-specific small molecule binders. In some embodiments, the presence or absence of p62, Ub, HDAC6, CFTR or VCP is determined by an antibody.

The antibodies of this disclosure bind specifically to a predictive or prognostic biomarker. The antibody can be prepared using any suitable methods known in the art. The antibody can be any immunoglobulin or derivative thereof, whether natural or wholly or partially synthetically produced. All derivatives thereof which maintain specific binding ability are also included in the term. The antibody has a binding domain that is homologous or largely homologous to an immunoglobulin binding domain and can be derived from natural sources, or partly or wholly synthetically produced. The antibody can be a monoclonal or polyclonal antibody. In some embodiments, an antibody is a single chain antibody. Those of ordinary skill in the art will appreciate that an antibody can be provided in any of a variety of forms including, for example, humanized, partially humanized, chimeric, chimeric humanized, etc. The antibody can be an antibody fragment including, but not limited to, Fab, Fab′, F(ab′)2, scFv, Fv, dsFv diabody, and Fd fragments. The antibody can be produced by any means. For example, the antibody can be enzymatically or chemically produced by fragmentation of an intact antibody and/or it can be recombinantly produced from a gene encoding the partial antibody sequence. The antibody can comprise a single chain antibody fragment.

Alternatively, or additionally, the antibody can comprise multiple chains which are linked together, for example, by disulfide linkages, and any functional fragments obtained from such molecules, wherein such fragments retain specific-binding properties of the parent antibody molecule. Because of their smaller size as functional components of the whole molecule, antibody fragments can offer advantages over intact antibodies for use in certain immunochemical techniques and experimental applications.

A detectable label can be used in the methods described herein for direct or indirect detection of the biomarkers when generating aggresome data in the methods of the quantification. A wide variety of detectable labels can be used, with the choice of label depending on the sensitivity required, ease of conjugation with the antibody, stability requirements, and available instrumentation and disposal provisions. Those skilled in the art are familiar with selection of a suitable detectable label or probe based on the assay for detection and quantification of the biomarkers in the methods of the present invention. Suitable detectable labels include, but are not limited to, Quantum dots, fluorescent dyes (e.g., fluorescein, fluorescein isothiocyanate (FITC), Oregon Green™, rhodamine, Texas red, tetrarhodimine isothiocynate (TRITC), Cy3, Cy5, Alexa Fluor®647, Alexa Fluor® 555, Alexa Fluor® 488), fluorescent markers (e.g., green fluorescent protein (GFP), phycoerythrin, etc.), enzymes (e.g., luciferase, horseradish peroxidase, alkaline phosphatase, etc.), nanoparticles, biotin, digoxigenin, metals, and the like.

The nanoparticle or quantum dot (QD) of this disclosure bind specifically to an antibody or antibodies to form immunoconjugates for specific binding and quantification of predictive or prognostic biomarker. These QD-immunoconjugates can be prepared using any suitable methods known to those skilled in the art such as a linker, antibody and QD reaction. The QD can be any fluorescent QD or derivative thereof, whether natural or wholly or partially synthetically produced. All derivatives thereof which maintain specific fluorescence activity are also included in the term. The QDs are nanoparticles having optical and electronic properties, as known to those skilled in art, QDs when illuminated by UV light, excited to a state of higher energy, where excited QD electrons emit variety of specific color fluorescence, as known in the art.

For mass-spectrometry-based analysis, differential tagging with isotopic reagents, e.g., isotope-coded affinity tags (ICAT) or the more recent variation that uses isobaric tagging reagents, iTRAQ (Applied Biosystems, Foster City, CA), followed by multidimensional liquid chromatography (LC) and tandem mass spectrometry (MS/MS) analysis can provide a further methodology in practicing the methods of this disclosure.

A chemiluminescence assay using a chemiluminescent antibody or nanoparticle can be used for sensitive, non-radioactive detection of proteins. An antibody labeled with fluorochrome also can be suitable. Examples of fluorochromes include spectrum of quantum dot based fluorescent probes, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), beta-galactosidase, urease, and the like. Detection systems using suitable substrates for horseradish-peroxidase, alkaline phosphatase, beta-galactosidase are well known in the art.

A signal from the direct or indirect label can be analyzed, for example, using a microscope, such as a fluorescence microscope or a fluorescence scanning microscope or FACS (FIG. 5(b) or point of care readers, disposable, or reusable readers (such as LEDx readers shown in FIGS. 6(a)-6(d)), etc.

Alternatively, a spectrophotometer can be used to detect color from a chromogenic or fluorescent substrate or a probe; a radiation counter to detect radiation such as a gamma counter for detection of ¹²⁵I; or a fluorometer to detect fluorescence in the presence of light of a certain wavelength. If desired, assays used to practice the methods of this disclosure can be automated or performed robotically, and the signal from multiple samples can be detected simultaneously.

Although a few embodiments of the present invention have been shown and described, it would be appreciated by those skilled in the art that changes may be made in this embodiment without departing from the principles and spirit of the invention, the scope of which is defined in the claims and their equivalents.

What is claimed is:

1. A method for predicting and diagnosing chronic obstructive pulmonary disorder (COPD)-emphysema and age-related lung disease in a subject, comprising:
(a) generating aggresome positive quantitative data from a respiratory sample, saliva or body fluid sample, obtained from the subject based on a quantitative and/or direct analysis comprising immunoprecipitation and quantum dot (QD) immunoconjugate(s) fluorescence, immunofluorescent or chemiluminescent staining signal or intensity, and/or morphological characteristics of peri-nucleated bodies in the cells of the sample, wherein aggresomes are identified in a context of a surrounding nucleus in cells and/or based on a combination of the immunofluorescent or chemiluminescent staining signal or intensity and/or morphological characteristics;
(b) obtaining baseline/clinical data or individual risk factors for the subject; and
(c) combining the aggresome positive quantitative data with the baseline/clinical data or risk factors of the subject or vice versa to predict, diagnose and/or validate COPD-emphysema, or age-related lung disease in the subject.

2. The method of claim 1, wherein the clinical data comprises one or more pieces of risk factors, imaging, lung function or pulmonary function test (PFT) and/or clinical history data.

3. The method of claim 2, wherein the imaging data is generated using a quantum dot (QD)/nanoparticle, contrast agent, molecular probe and/or aggresome dye-based positron emission tomography-computed tomography (PET/CT), X-ray fluoroscopy, CT or magnetic resonance imaging (MRI).

4. The method of claim 3, wherein the one or more pieces of imaging data are selected from the group consisting of maximum standardized uptake value (SUVₘₐₓ), maximum aggresome diameter, number and/or location.

5. The method of claim 2, wherein the lung function or PFT data is generated by spirometry, force oscillation technique (FOT), impulse oscillometry (IOS) or electrical impedance tomography (EIT).

6. The method of claim 5, wherein the COPD is a Global Initiative for Chronic Obstructive Lung Disease (GOLD) Stage I-IV emphysema and/or age-related lung condition.

7. The method of claim 2, wherein the one or more individual risk factors are selected from the group consisting of age, gender, ethnicity, lung disease history, smoking status, genetic predisposition, environmental and smoke or vapor exposure.

8. The method of claim 7, wherein the aggresome positive quantitative data and the clinical data comprise measurable features or at least one risk factor.

9. The method of claim 8, wherein the measurable features or risk factors are analyzed using a predictive model and/or utilize artificial intelligence, wherein the diagnosis is expressed as a risk score.

10. The method of claim 1, wherein the aggresome positive quantitative data is generated by lateral flow assay (LFA), imaging/microscopy, enzyme linked immunosorbent assay (ELISA) and/or flow cytometry, wherein the aggresome positive quantitative data is analyzed using an application (app) and/or analytical software.

11. The method of claim 10, wherein the microscopy provides a field of view comprising at least 5 punta-bodies as aggresomes surrounding nuclei, further comprising a step of obtaining an aggresome count for the sample using microscopy, or fluorescent scanning and quantification of aggresome count by flow cytometry, ELISA, or fluorescent microscopy.

12. The method of claim 10, wherein the fluorescent or chemiluminescent staining of nucleated cells of the sample comprises aggresome, p62 and ubiquitin, CFTR, or HDAC6 and/or Hoechst or diamidino-2-phenylindole (DAPI), wherein the aggresomes comprise distinct fluorescent or chemiluminescent staining, surrounding the nucleus in the cells of the sample.

13. The method of claim 10, wherein the aggresomes comprise distinct morphological characteristics compared to a surrounding nucleus or organelles in cells of the sample and/or the morphological characteristics comprising one or more of the groups consisting of aggresome size, aggresome shape, punta-body size, punta-body shape and aggresome to nuclear or cytoplasmic ratio.

14. The method of claim 1, further comprising an initial step of lysing cells in the sample and/or immunomagnetic separation of aggresome containing cells from the sample.

15. The method of claim 14, wherein the identification or quantification of the aggresomes further comprises determining change(s) in, p62, Ub, CFTR and/or HDAC6 immunoprecipitation, the quantum dots immunoconjugate(s) fluorescence or the immunofluorescent or chemiluminescent staining signal or intensity from background and baseline data using an LFA test.

16. The method of claim 15, further comprising obtaining a signal intensity for the sample on the LFA test under UV or another method of excitation of quantum dots (QDs) using an LFA reader, a camera, scanner or a spectrophotometer.

17. The method of claim 16, wherein the subject has >1.5-fold increase in aggresome levels from background or baseline data for predicting or validating the diagnosis, wherein levels increase exponentially with disease progression or severity of emphysema (Global Initiative for Chronic Obstructive Lung Disease (GOLD) stage I-IV).

18. The method of claim 17, wherein the diagnosis is expressed as a risk score for predicting COPD-emphysema followed by validation of disease severity, GOLD stage or prognosis for targeted intervention.

19. A device used as a reader for a lateral flow assay (LFA) test with test lines and quantum dot (QD) immunoconjugate(s), comprising:
(a) a body with an internal region, wherein the body is either U-shaped with side arms having magnets mounted inside of the side arms for immunomagnetic separation, or the body has a base, an upright section and the top arm, wherein an image sensor is mounted inside the top arm for capturing LFA images; and
(b) ultraviolet (UV) LED lights mounted on the top arm or the side arms of the body facing the internal region of the body;
(c) wherein when a respiratory sample, body fluid, tissue/cell or biological sample is run on the LFA test strips, the UV lights are turned on to excite the quantum dots at a 315-400 nm wavelength, and
(d) wherein the images of LFA test lines are captured by the image sensor for data analysis.

20. The device of claim 19, wherein:
(a) the body further comprises a processor, a rechargeable battery, and a power connection and/or charging port; and
(b) the image sensor is a smartphone or tablet for a home-based device or a camera/scanner for a point of care (POC) device.
